(19) United States
(12) Patent Application Publication
(10) Pub. No.: US 2014/0186467 A1
(43) Pub. Date: Jul. 3, 2014

(54) COMPOSITION AND USE FOR ERADICATION OF HIV, TREATMENT OF AIDS AND OTHER DISEASES INCLUDING TUBERCULOSIS IN A HUMAN

(71) Applicant: BNEERS CORP., Fort Washington, MD (US)

(72) Inventors: Comfort Wynee DIGGS, Monrovia (LR); Christian SOMUAH, Arlington, VA (US); Isaac Dowor JOEMAH, II, Washington, DC (US)

(73) Assignee: BNEERS CORP., Fort Washington, MD (US)

(21) Appl. No.: 14/197,629

(22) Filed: Mar. 5, 2014

Related U.S. Application Data
(63) Continuation-in-part of application No. PCT/US2013/056790, filed on Aug. 27, 2013.
(60) Provisional application No. 61/695,447, filed on Aug. 31, 2012.

(51) Int. Cl. A61K 33/26; A61K 31/295; A61K 47/44; A61K 31/43

(52) U.S. Cl. CPC A61K 33/26; A61K 31/43; A61K 31/295; A61K 47/44
USPC 424/648; 514/199

(57) ABSTRACT

A composition comprising at least one penicillin, at least one iron source, and at least one oil, method of making, and method of use thereof are described. Although suitable for many forms and uses, the composition is, in some embodiments, suitable as a pharmaceutical composition, and, in some embodiments, as an antiviral composition capable of restoring T cell count and eradicating HIV virus from patients suffering from HIV/AIDS and other diseases including Tuberculosis.

The present application is a continuation in part of PCT/US13/56790, filed Aug. 27, 2013, which application claims the priority benefits of U.S. Provisional application no. 61/695,447, filed Aug. 31, 2012, each application of which is incorporated by reference herein in its entirety.

The present description regards a composition, in particular a pharmaceutical composition comprising at least one penicillin, at least one iron source, and oil. Although suitable for many forms and uses, the composition is, in some embodiments, suitable as a pharmaceutical composition, and, in some embodiments, as an antiviral composition capable of treating HIV and Tuberculosis in a human.

HIV infection is a condition caused by the human immunodeficiency virus (HIV). The condition gradually destroys the immune system, which makes it harder for the body to fight infections.

Humans, including those infected with HIV, have T cells, which are a type of white blood cells (lymphocytes) making up part of the immune system. T cells help a body fight diseases or harmful substances. T cells are also called “CD4 cells” or “helper cells.”

Lower than normal T cell levels may be due to various ailments. For example, common ailments associated with T cell counts below 200 cells/mm³ include AIDS, AIDS dementia, and wasting syndrome caused by HIV itself. Similarly, a common ailment associated with T cell counts below 200 cells/mm³ include AIDS.

The present inventors know that when a human infected with HIV has a weakened immune system (<500 cells/mm³), an HIV antiviral medicine is sometimes administered to the infected human. The present inventors also know that in some cases, even before the human infected with HIV has a weakened immune system, an HIV antiviral medicine is administered to the infected human.

In some embodiments, a composition, comprises at least one penicillin; at least one iron source; and oil. Although suitable for many forms and uses, the composition is, in some embodiments, suitable as composition capable of treating HIV in a human having a weakened immune system (<500 cells/mm³) or the composition is, in some embodiments, suitable as composition capable of treating HIV in a human having a normal immune system (>500 cells/mm³) In some embodiments, a human having a weakened immune system below 200 cells/mm³ and AIDS

Additional results of the described embodiments will be set forth in part in the description which follows, and in part will be obvious from the description, or may be learned by practice of the embodiments disclosed herein. The results of practicing the embodiments will be realized and attained by means of the elements and combinations particularly pointed out in the appended claims.

It is to be understood that both the foregoing general description and the following detailed description are exemplary and explanatory only and are not restrictive of the invention, as claimed.

The accompanying description serves to explain the principles of the invention.

Reference will now be made in detail to the exemplary embodiments and examples.

Inventors named herein discovered a composition comprising, in part, natural ingredients extracted from plant materials, which ingredients when administered, made it possible to increase T cell counts by over 300% and eradicate HIV virus, cure AIDS and other diseases such as tuberculosis. The component parts were isolated, purified, and identified. The purified and isolated active components, apart from any extracting material, are described below.

In some embodiments, a composition, comprises at least one penicillin; at least one iron source; and at least one oil (“core composition”).

In some embodiments, the at least one penicillin is present in an amount ranging from 33.0 to 99.5% weight percent of the total weight of the at least one penicillin, at least one iron source, and at least one oil (i.e., the core composition). In some embodiments, the same weight percentage is from 45 to 90% weight percent or from 60 to 85% weight percent.

In some embodiments, at least one iron source is present in an amount ranging from 0.5 to 65.5% weight percent of the total weight of the core composition. In some embodiments, the same weight percentage is from 8.0 to 55% weight percent or from 14.0 to 40.0% weight percent.

In some embodiments, the at least one oil is present in an amount ranging from 0.00001 to 9.0% weight percent of the total weight of the core composition. In some embodiments, the same weight percentage is from 0.0002 to 7.0% weight percent or from 0.006 to 6.0% weight percent or from 0.01 to 1.0% weight percent.

The composition comprises at least one penicillin. In some embodiments, the at least one penicillin is chosen from Benzylpenicillin, Phenoxymethylpenicillin, Benzathine benzylpenicillin, Benzathine phenoxymethylpenicillin, Penicillin G, Penicillin G procaine, Penicillin V, Carfecillin, Ampicillin, Pivampicillin, Carbenicillin, Amoxicillin, Carindacillin, Bacampicillin, Pivmecillinam, Azlocillin, Mezlocillin, Piperacillin, Ticarcillin, Talampicillin, Sulbenicillin, Hetacillin, Propicillin, Pheneticillin, Dicloxacillin, Cloxacillin, Meticillin, Oxacillin, Flucloxacillin, Biapenem, Apalcillin, Aspoxicillin, Ciclacillin, Clemizole penicillin, Imipenem, Lenampicillin, Nafcillin, and Panipenem and pharmaceutically acceptable salts thereof.

In some embodiments, the at least one penicillin is chosen from Benzylpenicillin, Penicillin G benzathine, Penicillin G potassium, Penicillin G procaine, Penicillin G sodium, and Penicillin V.

In some embodiments, the at least one penicillin is chosen from Penicillin G procaine.

The at least one penicillin is present in the composition in an amount effective for decreasing viral load of HIV when administered with the at least one source of iron and the at least one oil. The effective dose depends on the age, weight, and administered form of composition. In some embodiments, the at least one penicillin is present in an amount ranging from 100 to 25,000 mg daily dose. In some embodiments, the at least one penicillin is present in an amount ranging from 400 to 18,000 mg daily dose or from 800 to 5,000 mg daily dose or from 1,500 to 4,000 mg daily dose. In some embodiments, the at least one penicillin is present in an amount ranging from 500 to 1,300 mg daily dose. In some embodiments, the amount is from 20 to 50 mg/kg daily dose or from 25 to 40 mg/kg daily dose.

In some embodiments, daily doses of the at least one penicillin are shown below in Table 1 for representative members of the at least one penicillin.

TABLE 1
Representative daily doses of penicillin

| | Low daily | High daily |
|---|---|---|
| Penicillin | (mg) | (mg) |
| amoxicillin | 1000 | 1750 |
| ampicillin | 2000 | 4000 |
| bacampicillin | 2000 | 4000 |
| carbenicillin | 2000 | 4000 |
| cloxacillin | 1000 | 2000 |
| dicloxacillin | 500 | 1000 |
| flucloxacillin | 1000 | 2000 |
| methicillin | 3000 | 4000 |
| mezlocillin | 18000 | 24000 |
| nafcillin | 1500 | 6000 |
| oxacillin | 3000 | 6000 |
| penicillin | 500 | 1250 |
| G/penicillin G | | |
| procaine | | |
| benzathine | 750 | 1500 |
| penicillin G | | |
| K penicillin | 3750 | 18000 |
| G/Na | | |
| penicillin G | | |
| penicillin V | 500 | 2000 |
| piperacillin | 18000 | 24000 |
| pivampicillin | 1050 | 2100 |
| pivmecillinam | 400 | 800 |
| ticarcillin | 16000 | 18000 |

The at least one source of iron is chosen from pharmaceutically acceptable forms of elemental, ferrous, and ferric moieties. In some embodiments, the at least one iron source is chosen from ferrous fumarate, ferrous sulfate, and ferrous gluconate. In some embodiments, the at least one iron source is chosen from iron carbonyl, iron polysaccharide, iron protein succinylate, and heme iron polypeptides. In some embodiments, the at least one iron source is chosen from iron dextran, iron sucrose, iron carboxymaltose, ferrous glycine sulfate, and ferroglycine sulfate.

The at least one iron source is present in the composition in an amount effective for decreasing viral load of HIV when administered with the at least one penicillin and at least one oil. Elemental iron is the amount of iron form that is available for absorption. In some embodiments, the at least one iron source is present in an amount effective to deliver a daily dose ranging from 100 to 750 mg of elemental iron. In some embodiments, the at least one iron source is present in an amount effective to deliver a daily dose ranging from 150 to 600 mg of elemental iron. In some embodiments, the at least one iron source is present in an amount effective to deliver a daily dose from 12 to 18 mg/kg of elemental iron.

In some embodiments, daily doses of the at least one iron source are shown below in Table 2 for representative members of the at least one iron source.

TABLE 2
Representative daily doses of the iron source.

| | | | | Low | High |
|---|---|---|---|---|---|
| | Low | High | | daily | daily |
| | daily | daily | Percent | elemental | elemental |
| | dose | dose | elemental | Fe dose | Fe dose |
| Iron source | (mg) | (mg) | Fe | (mg) | (mg) |
| ferrous furmate | 325 | 1000 | 33% | 107 | 330 |
| ferrous sulfate | 500 | 1000 | 20% | 100 | 200 |
| ferrous gluconate | 1250 | 1750 | 12% | 150 | 210 |
| polysaccharide- | 150 | 300 | 100% | 150 | 300 |
| iron | | | | | |
| heme-iron | 175 | 350 | 100% | 175 | 350 |
| polypeptide | | | | | |
| ferric ammonium | 1000 | 2000 | 18% | 180 | 360 |
| citrate | | | | | |
| Ferrous | 1000 | 2000 | 20% | 200 | 400 |
| biglycinate | | | | | |

Any oil which is pharmaceutically acceptable is usable as the at least one oil. In some embodiments, the at least one oil is a vegetable oil. In some embodiments, the at least one oil is an animal oil. In some embodiments, the at least one oil is chosen from oleoresins (solvent-free) and natural extractives (including distillates).

In some embodiments, the at least one oil is chosen from oil derived from palm, soybean, rapeseed, canola, sunflower seed, peanut, cottonseed, palm kernel, coconut, olive, safflower oil, corn, grape seed, hazelnut, linseed, flax seed, rice bran, castor bean, and sesame oil. In some embodiments, the oil is chosen from olive, corn, peanut, nut, soy, rapeseed, cottonseed, vitamin E, fish, or tallow-derived oils, and mineral oils. In some embodiments, the oil is chosen from almond oil, apricot kernel oil PEG-6 esters, canola oil, castor oil, cedar leaf oil, cinnamon oil, clove oil, coconut oil, coriander oil, corn oil, corn oil PEG-6 esters, cottonseed oil, eucalyptus oil, fractioned coconut oil, lemon oil, light mineral oil, lime oil, nutmeg oil, olive oil, orange oil, polyoxyl castor oils, poppy seed oil, sesame oil, and soybean oil. In some embodiments, the oil is chosen from hydrogenated oils, e.g., hydrogenated castor oil, hydrogenated palm oil, and hydrogenated soybean oil. Exemplary embodiments of the at least one oil are commercially available.

The at least one oil is present in the composition in an amount effective for decreasing viral load of HIV/AIDS when administered with the at least one penicillin and at least one iron source. In some embodiments, the at least one oil is present in an amount ranging from 0.0005 to 50 mg daily dose. In some embodiments, the same amount is from 0.001 to 25 mg daily dose or from 0.003 to 15 mg daily dose or from 0.005 tol mg daily dose. In some embodiments, daily doses of the at least one oil are shown below in Table 3 for representative members of the at least one oil.

TABLE 2
Representative daily doses of the oil.

| Oil | Low amount (mg) | High amount (mg) |
|---|---|---|
| Sunflower | 0.0005 | 25 |
| Sesame | 0.003 | 1 |
| Fish | 0.005 | 15 |
| Soybean | 0.001 | 50 |
| hydrogenated palm | 0.0005 | 0.003 |
| Castor bean | 0.0005 | 50 |

In some embodiments, the composition comprises any combination of one ingredient from Table 1, one ingredient from Table 2, and one ingredient from Table 3. In some embodiments, the amounts vary due to the dosing regimen. For example, if there are an integer “n” doses per day, then the amounts would vary by (1/n) times those amounts shown in Tables 1-3. For example, typical values of n range from 1, 2 . . . 6. If n=3, then the values of the amounts for each ingredient in Tables 1-3 is divided by 3.

In some embodiments, the composition is in a pharmaceutically acceptable vehicle, i.e., a carrier or inert medium used as a solvent (or diluent) in which the core composition is formulated and/or administered.

In some embodiments, the composition further comprises caffeine or a salt thereof. In some embodiments, the caffeine is in the form of a salt, such as caffeine citrate. In some embodiments, the sodium chloride is present in the composition in an amount ranging from 10 to 200 mg daily dose. In some embodiments, the amount is from 20 to 150 mg daily dose or from 60 to 100 mg daily dose.

In some embodiments, the composition further comprises maltase. Maltase, according to the MeSH Headings, is part of the alpha-Glucosidases, i.e., enzymes that catalyze the exohydrolysis of 1,4-alpha-glucosidic linkages with a release of alpha-glucose. Maltase has a CAS Type 1 Name of alpha-D-Glucoside glucohydrolases and an Enzyme Commission EC Registry Number of EC 3.2.1.20. Maltase is commercially available.

In some embodiments, the maltase is in the form of a lyophilized powder having greater than 125 or greater than 50 units/mg protein. Other forms are possible.

In some embodiments, the maltase is present in the composition in an amount ranging from 1 to 100 mg daily dose. In some embodiments, the amount is from 5 to 50 mg daily dose or from 10 to 25 mg daily dose.

In some embodiments, the composition further comprises pharmaceutically acceptable sodium. In some embodiments, the sodium is in the form of sodium chloride. In some embodiments, the sodium chloride is present in the composition in an amount ranging from 1,500 to 3,000 mg daily dose. In some embodiments, the amount is from 1,250 to 2,750 mg daily dose or from 1,500 to 2,300 mg daily dose.

In some embodiments, the composition comprises a pharmaceutically acceptable excipient. Pharmaceutically acceptable excipients include substances added to a core composition in order to provide suitable consistency to the dosage form. Pharmaceutically acceptable excipients include binders, matrix, base or diluent in pills, tablets, creams, salves, and the like. In some embodiments, the pharmaceutically acceptable excipient is inert.

Techniques for formulation and administration of drugs and pharmaceutical compositions are found in “Remington: The Science and Practice of Pharmacy,” Lippincott Williams & Wilkins, 21^{st} ed. (2005).

In some embodiments, the pharmaceutical composition is made by processes well known in the art, e.g., by means of conventional mixing, dissolving, granulating, dragee-making, levigating, emulsifying, encapsulating, entrapping, lyophilizing processes or spray drying.

In some embodiments, the pharmaceutical composition is made by bringing in association the at least one penicillin, at least one source of iron, at least one oil with a pharmaceutical vehicle. In some embodiments, the pharmaceutical composition further comprises one or more ingredients chosen form a pharmaceutically acceptable vehicle, sodium, maltase, caffeine, and other pharmaceutically acceptable excipients.

In some embodiments, the pharmaceutical composition has a form and component ingredients dependent upon the route of administration chosen and the patient chosen.

In some embodiments, the pharmaceutical composition is made in a conventional manner using one or more physiologically acceptable carriers comprising excipients which facilitate processing of core composition into preparations which are pharmaceutically useable.

In some embodiments, the dosage form is chosen from tablets, troches, dispersions, suspensions, solutions, capsules, patches, syrups, elixirs, gels, powders, magmas, lozenges, ointments, creams, pastes, plasters, lotions, discs, suppositories, nasal sprays, oral sprays, aerosols, and the like.

In some embodiments, the pharmaceutical composition is in a form suitable for injection. In some embodiments, forms suitable for injection are chosen from aqueous solutions, such as those in physiologically compatible buffers such buffers, optionally, having a stabilizing amount of surfactant or co-solvent, or physiological saline buffer.

In some embodiments, the pharmaceutical composition is in a form suitable for oral administration. In some embodiments, the pharmaceutical composition is made by combining the core composition with at least one pharmaceutically acceptable vehicle. Such pharmaceutically acceptable vehicles facilitate forming tablets, pills, lozenges, dragees, capsules, liquids, gels, syrups, slurries, suspensions and the like, for oral ingestion by a patient. In some embodiments, the pharmaceutical composition is made using a solid excipient, optionally grinding the resulting mixture, and processing the mixture of granules, after adding other excipients if desired, to obtain tablets or dragee cores. In some embodiments, excipients are chosen from fillers such as sugars, including lactose, sucrose, mannitol, and sorbitol; cellulose preparations such as, for example, maize starch, wheat starch, rice starch, and potato starch; and other materials such as gelatins, gum tragacanth, methyl cellulose, hydroxypropylmethyl-cellulose, sodium carboxymethylcellulose, and polyvinyl-pyrrolidone (PVP). In some embodiment, the pharmaceutical composition comprises a disintegrating agent, such as those chosen from cross-linked polyvinyl pyrrolidone, agar, and alginic acid. In some embodiments, the pharmaceutical composition comprises a salt, such as sodium alginate.

In some embodiments, the pharmaceutical composition is in the form of a dragee. Dragee cores are provided with suitable coatings. In some embodiments, concentrated sugar solutions are used which optionally comprise gum arabic, talc, polyvinyl pyrrolidone, carbopol gel, polyethylene glycol, and/or titanium dioxide, lacquer solutions, and suitable organic solvents or solvent mixtures. Dyestuffs or pigments, in some embodiments, are added to the tablets or dragee coatings for identification or to characterize different combinations of active compound doses.

In some embodiments, the pharmaceutical composition is in the form of a capsule, such as those chosen from oral push-fit capsules comprising gelatin; and soft, sealed capsules comprising gelatin and a plasticizer, such as glycerol or sorbitol. In some embodiments, the push-fit capsules comprise the core composition in admixture with a filler, such as lactose, a binder, such as starch, and/or a lubricant, such as talc or magnesium stearate and, optionally, one or more stabilizers. In some embodiments, the soft-capsules comprise the core composition dissolved or suspended in one or more suitable liquids, such as fatty oils, liquid paraffin, liquid polyethylene glycols, cremophor, capmul, medium or long chain mono- di- or triglycerides; and optionally one or more stabilizers.

In some embodiments, the pharmaceutical composition is in a form suitable for parenteral administration, e.g., by bolus injection or continuous infusion. In some embodiments, formulations suitable for injection are in unit dosage form, e.g., in ampoules or in multi-dose containers, and optionally further comprising at least one preservative. In some embodiments, the pharmaceutical composition is in a form chosen from suspensions, solutions, or emulsions in oily or aqueous pharmaceutical vehicles, and optionally further comprise one or more excipients such as suspending agents, stabilizing agents, and dispersing agents.

In some embodiments, the pharmaceutical composition comprises an excipient chosen from sodium carboxymethyl cellulose, sorbitol, and dextran.

In some embodiments, the pharmaceutical composition is in the form of a powder. In some embodiments, the powder is constituted with a suitable pharmaceutical vehicle, e.g., sterile, pyrogen-free water, before administering.

In some embodiments, the pharmaceutical composition is in the form of a depot. In some embodiments, the depot is administered by implantation (for example, subcutaneously or intramuscularly) or by intramuscular injection.

In some embodiments, the pharmaceutical composition comprises a pharmaceutical vehicle chosen from a co-solvent system comprising benzyl alcohol, a nonpolar surfactant, a water-miscible organic polymer and an aqueous phase such as the VPD co-solvent system. VPD is a solution of 3% W/v benzyl alcohol, 8% W/v of the nonpolar surfactant Polysorbate 80, and 65% W/v polyethylene glycol 300, made up to volume in absolute ethanol. In some embodiments, the proportions of such a co-solvent system are varied without destroying its solubility and toxicity characteristics. Furthermore, the identity of the co-solvent components, in some embodiments, is varied: for example, in some embodiments, other low-toxicity nonpolar surfactants are substituted for Polysorbate 80.

In some embodiments, the pharmaceutical composition comprises suitable solid or gel phase pharmaceutical vehicles and/or excipients. Examples of such vehicles or excipients include, but are not limited to, calcium carbonate, calcium phosphate, various sugars, starches, cellulose derivatives, gelatin, and polymers such as polyethylene glycols.

In some embodiments, the composition further comprises at least one excipient present in an amount sufficient to perform its function. In some embodiments, the at least one excipient is added by mixing the at least one excipient with the core composition.

In some embodiments, the at least one excipient is chosen from preservatives, sweeteners, color additives, flavors, nutrients, and pH control agents and/or acidulants.

In some embodiments, a preservative is present in the composition in an amount sufficient to prevent spoilage from bacteria, molds, fungi, or yeast (antimicrobials); to slow or prevent changes in color, flavor, or texture and delay rancidity (antioxidants); or to maintain freshness. Exemplary preservatives include, e.g., ascorbic acid, citric acid, sodium benzoate, calcium propionate, sodium erythorbate, sodium nitrite, calcium sorbate, potassium sorbate, BHA, BHT, EDTA, and tocopherols (Vitamin E).

In some embodiments, a sweetener is present in the composition in an amount sufficient to affect sweetness. Exemplary sweeteners include, e.g., sucrose (sugar), glucose, fructose, sorbitol, mannitol, corn syrup, high fructose corn syrup, saccharin, aspartame, sucralose, acesulfame potassium (acesulfame-K), and neotame.

In some embodiments, a color additive is present in the composition in amount sufficient to offset color loss due to exposure to light, air, temperature extremes, moisture and storage conditions; to correct natural variations in color; to enhance colors that occur naturally; or to provide color. Exemplary color additives include, e.g., FD&C Blue Nos. 1 and 2, FD&C Green No. 3, FD&C Red Nos. 3 and 40, FD&C Yellow Nos. 5 and 6, Orange B, Citrus Red No. 2, annatto extract, beta-carotene, grape skin extract, cochineal extract or carmine, paprika oleoresin, caramel color, fruit and vegetable juices, and saffron.

In some embodiment, a flavor is present in the composition in an amount sufficient to add a specific flavor. Exemplary flavors and/or spices include, e.g., natural flavoring, artificial flavor, and spices.

In some embodiments, a nutrient is present in the composition in an amount sufficient to add nutrients that are lacking in the diet (fortification). Exemplary nutrients include, e.g., thiamine hydrochloride, riboflavin (Vitamin B2), niacin, niacinamide, folate or folic acid, beta carotene, potassium iodide, iron or ferrous sulfate, alpha tocopherols, ascorbic acid, Vitamin D, amino acids (L-tryptophan, L-lysine, L-leucine, L-methionine).

In some embodiments, a pH control agent and/or an acidulant is/are present in the composition in an amount sufficient to control acidity and alkalinity and/or to prevent spoilage. Exemplary pH control agents and/or acidulants include, e.g., lactic acid, citric acid, ammonium hydroxide, and sodium carbonate.

In some embodiments, the composition is in the form of a tablet or capsule. In some embodiments, a tablet comprises a mixture of core ingredients and one or more excipients, usually in powder form, pressed or compacted from a powder into a solid dose. The excipients, in some embodiments, are chosen from color additives, flavors and spices, flavor enhancers, preservatives, binders, fillers, and lubricants. The color additives, flavors and spices, flavor enhancers, and preservatives were noted above.

In some embodiments, a binder is present in the composition in an amount sufficient to produce uniform texture, improve “mouth-feel” or binding of the tablet or capsule ingredients. Exemplary binders include, e.g., gelatin, pectin, guar gum, carrageenan, xanthan gum, and whey.

In some embodiments, a filler is present in the composition in an amount sufficient to bulk up the tablet or capsule Exemplary binders include, e.g., sugars, such as lactose, sucrose, mannitol, calcium carbonate, and calcium phosphate.

In some embodiments, a lubricant is present in the composition in an amount sufficient to prevent clumping of the ingredients of the tablet or capsule Exemplary lubricants include, e.g., talc, silica, stearic acid, and magnesium stearate.

In some embodiments, the pharmaceutical composition is administered via a route chosen from oral, intraoral, rectal, transmucosal, intestinal routs; intramuscular, epicutaneous, parenteral, subcutaneous, transdermal, intramedullary, intrathecal, direct intraventricular, intravenous, intravitreal, intraperitoneal, intranasal, intramuscular, intradural, intrarespiratory, nasal inhalation or intraocular injections. In some embodiments, routes of administration are chosen from oral routes and parenteral routes.

The amount of the core composition (and any carrier or other ingredient) administered will be dependent on the subject being treated, the severity of the disorder or condition, the rate of administration, the disposition of the pharmaceutical composition, and the discretion of the prescribing physician.

In some embodiments, the dosage levels (of the core composition) below the lower limit of the above range is sufficient, while in other cases a larger dosage is sufficient.

In some embodiments, larger doses (of the pharmaceutical composition) are typically divided into several smaller doses for administration periodically throughout the day.

In some embodiments, the composition is administered to a subject. The subject is an animal, e.g., a human infected with HIV. In some embodiments, the human has AIDS.

Two types of HIV have been characterized: HIV-1 and HIV-2. Unless otherwise indicated, the term HIV refers to both HIV-1 and HIV-2.

In some embodiments, the human is infected with HIV-1. In some embodiments, the human is infected with HIV-2.

In some embodiments, the human infected with HIV has a T cell count below 500 cells/mm³ In some embodiments, the human infected with HIV has a T cell count greater than or equal to 500 cells/mm³ In some embodiments, the human infected with HIV has a T cell count below 200 cells/mm³ or AIDS.

In some embodiments, the human infected with HIV has one or more symptoms associated with AIDS (e.g., diarrhea, fatigue, fever, frequent vaginal yeast infections, headache, mouth sores, including yeast infection (thrush), muscle stiffness or aching, flashes of different types, including seborrheic dermatitis and psoriasis, sore throat, swollen lymph glands). In some embodiments, the human infected with HIV is asymptomatic of conditions associated with AIDS.

In some embodiments, the composition is administered to a human in need of enhancing of T cells and decreasing HIV viral load. In some embodiments, the human is infected with HIV. In some embodiments, the human has AIDS. In some embodiments, administering the composition makes it possible to eradicate the viral load in the human having HIV infection and Tuberculosis disease.

In some embodiments, the human infected with HIV has AIDS dementia, wasting syndrome caused by HIV itself.

In some embodiments, the composition is administered orally, parenterally, topically, rectally, nasally, buccally, vaginally, or via an implanted reservoir in dosage formulations. The term parenteral as used herein includes subcutaneous, intravenous, intramuscular, intraperitoneal, intrathecal, intraventricular, intrasternal or intracranial injection and infusion techniques.

In some embodiments, the composition is administered from one to six times a day for a period of time ranging from one week to one year. In some embodiments, the composition is administered one to four times a day for a period of time ranging from two weeks to six months. In some embodiments, the composition is administered two to three times a day for a period of time ranging from two to eight weeks or six to eight weeks.

In some embodiments, administering is for a period of time sufficient to remove at least 50% of the detectable amounts of HIV in the patient. The detectable amount is determined by measuring the detectable amount of HIV present before treatment (HIVᵢₙᵢₜᵢₐₗ) and during treatment (HIV_{final}) and thereafter calculating the decrease according to formula (1).

(HIV_{final}/HIVᵢₙᵢₜᵢₐₗ−1)×100% (1)

In some embodiments, administering is for a period of time sufficient to remove at least 60% of the detectable amounts of HIV in the patient. In some embodiments, administering is for a period of time sufficient to remove at least 70% of the detectable amounts of HIV in the patient. In some embodiments, administering is for a period of time sufficient to remove at least 80% of the detectable amounts of HIV in the patient. In some embodiments, administering is for a period of time sufficient to remove at least 90% of the detectable amounts of HIV in the patient. In some embodiments, administering is for a period of time sufficient to remove at least 95% of the detectable amounts of HIV in the patient. In some embodiments, administering is for a period of time sufficient to remove at least 99% of the detectable amounts of HIV in the patient. In some embodiments, administering is for a period of time sufficient to remove at least 99.9% of the detectable amounts of HIV in the patient. In some embodiments, administering is for a period of time sufficient to remove at least 100% of the detectable amounts of HIV in the patient. In other words, in some embodiments, administering the composition described herein makes it possible to not only minimize but also eliminate or eradicate the detectable amount of HIV/AIDS in the patient.

The present inventors have concluded that the composition described herein has antiviral activity against HIV or activity against AIDS and other diseases including Tuberculosis. The present inventors have concluded that the composition described herein makes it possible to cure AIDS. The present inventors have concluded that the composition described herein makes it possible to cure HIV.

### EXAMPLE 1

A scaled up set of amounts sufficient to result in a dosage form having 125 mg of penicillin G and 325 mg ferrous fumarate are mixed while adding 0.001 mg of vegetable oil. The resultant composition is suitable for mixing with an excipient like microcrystalline cellulose to form tablets; calcium carbonate or magnesium carbonate to form a chewable tablets; or mannitol, sorbitol, fructose, and/or maltose to form a chewable tablet. Alternatively, the resultant composition is capable of forming an aqueous suspension with or without a dispersing agent like soybean oil or PEG 400. The resultant formulation is suitable to be administered, e.g., from 3 to 6 times a day.

### Example 2

300 g of penicillin G procaine and 600 g ferrous sulfate are mixed while adding 1 mg of castor oil. The resultant composition (A) is suitable for forming the various dosage forms outlined in example 1.

Alternatively, to the resultant composition (A), 30 g of caffeine are mixed and the modified resultant composition (B) is suitable for forming the various dosage forms outlined in example 1.

The resultant formulation (A) or (B) is suitable to be administered, e.g., from 1 to 2 times a day. Administration, in some embodiments, is to those in need of bowel relaxation.

### Example 3

A scaled up set of amounts sufficient to result in a dosage form having 500 mg of penicillin V potassium salt and 1000 mg ferrous sulfate are mixed while adding 1 mg of palm oil.

The resultant composition (C) is suitable for forming the various dosage forms outlined in example 1.

Alternatively, to the resultant composition (C), 10 mg per dose of maltase (125 units/mg lyophilized powder) are mixed and the modified resultant composition (D) is suitable for forming the various dosage forms outlined in example 1.

The resultant formulation (C) or (D) is suitable to be administered, e.g., from 2 to 4 times a day. Administration, in some embodiments, is to those in need of digestive assistance.

### Example 4

In a manner similar to that used in Example 1, a dosage form having 1,000 mg of penicillin V sodium salt, 1000 mg ferrous sulfate, 0.001 mg of castor oil, and 750 mg of sodium chloride is made.

The formulation is administered in an amount of one dose twice a day to a human patient infected with HIV. The human patient is infected with HIV and has a T cell count below 500 cells/mm³.

Administration, in some embodiments, is to a human patient infected with HIV and having a T cell count greater than or equal to 500 cells/mm³.

### Example 5

In a manner similar to that used in Example 1, a dosage form having 700 mg of penicillin G procaine, 350 mg of elemental iron, 0.0005 mg of soybean oil, 600 mg of sodium chloride, 25 mg of caffeine citrate, and 10 mg of maltase (about 50 units/mg lyophilized powder) is made.

The formulation is administered in an amount of one dose twice a day to a human suffering from tuberculosis, HIV, and AIDS. The human patient is infected with HIV and has a T cell count below 500 cells/mm³ Administering according to this regimen is continued for a period of time ranging from one week to one year. Administration, in some embodiments, is to those in need of lesion relief, digestive assistance, and colon relaxation.

Administration, in some embodiments, is to a human patient infected with HIV and having a T cell count greater than or equal to 500 cells/mm³.

### Example 6

In a manner similar to that used in Example 1, a dosage form having 350 mg of penicillin G benzatine, 400 mg of elemental iron, 0.005 mg of castor oil, 600 mg of sodium chloride, 25 mg of caffeine citrate, and 10 mg of maltase (about 50 units/mg lyophilized powder) is made.

The formulation is administered via injection in an amount of one dose once a day to a human suffering from tuberculosis. Administering according to this regimen is continued for a period of time ranging from one week to one year. Administration, in some embodiments, is to those in need of lesion relief, digestive assistance, and colon relaxation.

### Example 7

### Use

In a manner similar to that used in Example 1, a dosage form having 150 mg of penicillin G procaine, 250 mg of elemental iron, 0.1 mg of castor oil and 0.1 soybean oil is made into a suspension.

The formulation is administered orally in an amount of one dose once a day to a human suffering from wasting syndrome caused by HIV. Administering according to this regimen is continued for a period of time ranging from one week to one month.

### Example 8

A composition comprising natural ingredients extracted from plant materials was administered to patients having AIDS and Tuberculosis, made it possible to increase T cell counts by over 300% and eradicate detectable amounts of the HIV virus. The component parts were isolated from the extracting material, purified, and identified. The component parts were screened for their activity.

### Example 9

Based on these examples and information known to each inventors, it is concluded that the composition described herein has antiviral activity against HIV and/or Tuberculosis. Based on these examples, it is concluded that administering the composition described herein makes it possible to minimize, if not eradicate, the detectable amount of HIV and/or Tuberculosis in the patient. Based on these examples, it is concluded that administering the composition described herein makes it possible to cure HIVAIDS and/or Tuberculosis in the patient. Based on these examples and information known to each inventors, the present inventors have concluded that the composition described herein makes it possible to cure AIDS. Based on these examples and information known to each inventors, the present inventors have concluded that the composition described herein makes it possible to cure HIV.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of curing HIV/AIDS and other diseases including Tuberculosis in a human patient in need thereof, comprising, administering, to a human patient infected with HIV/AIDS and/or Tuberculosis, a pharmaceutical composition capable of restoring T cell count and eradicating HIV virus from patients affected by HIV/AIDS and other diseases including Tuberculosis, comprising:
at least one penicillin;
at least one iron source; and
at least one oil.

2. The method of claim 1, wherein the human patient has a T cell count below 500 cells/mm³.

3. The method of claim 1, wherein the human patient has a T cell count greater than or equal to 500 cells/mm³.

4. The method of claim 1, wherein the human patient has an ailment chosen from AIDS, AIDS dementia, wasting syndrome caused by HIV itself.

5. The method of claim 1, wherein the human patient is asymptomatic of conditions associated with AIDS.

6. The method of claim 1, wherein the composition further comprises caffeine.

7. The method of claim 1, wherein the composition further comprises maltase.

8. The method of claim 1, wherein the composition further comprises sodium.

9. The method of claim 1, wherein the composition further comprises a pharmaceutically acceptable vehicle.

10. The method of claim 1, wherein the at least one penicillin is chosen from Benzylpenicillin, Phenoxymethylpenicillin, Benzathine benzylpenicillin, Benzathine phenoxymethylpenicillin, Penicillin G, Penicillin G procaine, Penicillin V, Carfecillin, Ampicillin, Pivampicillin, Carbenicillin, Amoxicillin, Carindacillin, Bacampicillin, Pivmecillinam, Azlocillin, Mezlocillin, Piperacillin, Ticarcillin, Talampicillin, Sulbenicillin, Hetacillin, Propicillin, Pheneticillin, Dicloxacillin, Cloxacillin, Meticillin, Oxacillin, Flucloxacillin, Biapenem, Apalcillin, Aspoxicillin, Ciclacillin, Clemizole penicillin, Imipenem, Lenampicillin, Nafcillin, and Panipenem and pharmaceutically acceptable salts thereof.

11. The method of claim 1, wherein the at least one penicillin is chosen from Benzylpenicillin, Penicillin G benzathine, Penicillin G potassium, Penicillin G procaine, Penicillin G sodium, and Penicillin V.

12. The method of claim 1, wherein the at least one penicillin is Penicillin G procaine.

13. The method of claim 1, administering is for a period of time sufficient to remove at least 90% of the detectable amounts of HIV and/or Tuberculosis in the patient.

14. The method of claim 1, wherein the administering is for a period of time sufficient to remove detectable amounts of HIV and or Tuberculosis in the patient.

15. A method of treating HIV and Tuberculosis in a human patient in need thereof, comprising, administering, to a human patient infected with HIV and or Tuberculosis and having a T cell count greater than or equal to 500 cells/mm³, an effective amount of a composition, comprising:
at least one penicillin;
at least one iron source; and
at least one oil
wherein the administering is for a period of time sufficient to remove at least 95% of the detectable amounts of HIV and/or Tuberculosis in the human patient.

16. The method of claim 15, wherein the administering is for a period of time sufficient to eradicate detectable amounts of HIV, cure AIDS and/or cure Tuberculosis in the human patient.

17. The method of claim 15, wherein the at least one penicillin is chosen from Benzylpenicillin, Penicillin G benzathine, Penicillin G potassium, Penicillin G procaine, Penicillin G sodium, and Penicillin V.

18. A method of treating HIV and Tuberculosis in a human patient in need thereof, comprising, administering, to a human patient infected with HIV and/or Tuberculosis and having a T cell count below 500 cells/mm³, an effective amount of a composition, comprising:
at least one penicillin;
at least one iron source; and
at least one oil
wherein the administering is for a period of time sufficient to remove at least 95% of the detectable amounts of HIV and/or Tuberculosis in the human patient.

19. The method of claim 18, wherein the administering is for a period of time sufficient to remove detectable amounts of HIV and or Tuberculosis in the human patient.

20. The method of claim 18, wherein the at least one penicillin is chosen from Benzylpenicillin, Penicillin G benzathine, Penicillin G potassium, Penicillin G procaine, Penicillin G sodium, and Penicillin V.
